(19) United States
(12) Patent Application Publication
(10) Pub. No.: US 2006/0134060 A1
(43) Pub. Date: Jun. 22, 2006

(54) Ophthalmic compositions comprising Aloe Vera

(76) Inventor: Erning Xia, Penfield, NY (US)

Correspondence Address: Bausch & Lomb Incorporated, One Bausch & Lomb Place, Rochester, NY, 14604-2701, US

(21) Appl. No.: 11/301,378

(22) Filed: Dec. 13, 2005

Related U.S. Application Data
(60) Provisional application No. 60/638,735, filed on Dec. 22, 2004.

(51) Int. Cl. A61K 8/97; A61K 36/886

(52) U.S. Cl. 424/74; 424/744

(57) ABSTRACT

Disclosed herein are new formulations containing Aloe Vera. These formulations are useful as eye drops that provide high patient compliance as well as carriers for pharmaceuticals such as steroids and anti-allergy ophthalmic drugs.

### CROSS REFERENCE

This application claims the benefit of Provisional Patent Application No. 60/638,735 filed Dec. 22, 2005 and is incorporated herein by reference.

Disclosed herein are new formulations containing Aloe Vera. These formulations are useful as eye drops that provide high patient compliance as well as carriers for pharmaceuticals such as steroids and anti-allergy ophthalmic drugs.

The use of Aloe Vera in ophthalmic formulations is known. For example, U.S. Pat. No. 4,788,007 to Baron discloses a method of using ophthalmic dosage of aqueous Aloe Vera as emollient and absorbent of UV radiation to shield eye retina by topically applying said gel in the eye; U.S. Pat. No. 6,013,259 to de de la Pena, et al. relates to a process for obtaining Aloe Vera gel and the ophthalmic use of Aloe Vera gel in the treatment of dry eye syndrome; and U.S. Pat. No. 6,573,299 relates to a method for the prevention and treatment of orbital disorders associated with the aging eye in mammals by the application of a topical composition comprising a permeation enhancing amount of one or more penetration enhancers, and one or more bio-affecting agent which penetrate into the underlying tissues and into the vascular network of the orbit.

Despite these various patents, there is still a need for new ophthalmic formulations that offer higher patient compliance and greater universal appeal than traditional dry eye products. This is accomplished by using natural Aloe Vera with various polymer systems in a well-buffered solution. The formulation provides customers with an enhanced dry eye relief and other physiological benefits.

Disclosed herein are ophthalmic compositions comprising Aloe Vera Gel preserved with the biguanide preservative Alexidine hydrochloride. It will be undisclosed that other ophthalmic preservatives such as those generally known to those of skill in the art may also be used to provide formulations in accordance with this invention. Such preservatives include benzalkonium chloride (BAK), sorbic acid/EDTA, H2O2 systems, Zinc preservative systems and stabilized hypochlorite preparations such as PURITE.

Aloe Vera is a nutritional storehouse found naturally. Aloe Vera has a long history as a multipurpose folk remedy. Commercially, it can be separated into two products: gel and latex. Aloe Vera gel is the leaf pulp or mucilage and is a thin clear jelly-like substance that contains vitamin B1, B2, B6, C; amino acids, and carbohydrate polymers such as mucopolysaccharides, glucomannans or pectic acid plus various other organic and inorganic compounds. Aloe Vera also works as an antioxidant and can be purchased from DSM Nutritional Products, Inc.

It is known that Aloe Vera can accumulate in the anterior segment and moisturizes surrounding ocular tissues when administered topically. Aloe Vera also scavenges free radicals and other damaging compounds in the ocular tissues that alter cell membranes, tamper with DNA, and even cause cell death. Therefore, the present composition may protect ocular tissues from the damage of free radicals and improve the general health of the eye. Aloe vera gel may be present in a composition according to the invention in amounts ranging from about 1 to about 50 percent by weight.

In addition to Aloe vera gel, the compositions of the invention herein also contain ophthalmically acceptable buffers such as Sodium borate/Boric acid. Other ophthalmically acceptable buffers suitable for use in the invention herein will be apparent to one having ordinary skill in the art.

The composition further comprises ophthalmic demulcents such as any of the following, within the established concentrations for each ingredient: (a) Cellulose derivatives: (1) Carboxymethylcellulose sodium, 0.2 to 2.5 percent, (2) Hydroxyethyl cellulose, 0.2 to 2.5 percent, (3) Hypromellose, 0.2 to 2.5 percent and (4) Methylcellulose, 0.2 to 2.5 percent; (b) Dextran 70, 0.1 percent when used with another polymeric demulcent agent in this section; (c) Gelatin, 0.01 percent; (d) Polyols, liquids such as (1) Glycerin, 0.2 to 1 percent, (2) Polyethylene glycol 300, 0.2 to 1 percent, (3) Polyethylene glycol 400, 0.2 to 1 percent, (4) Polysorbate 80, 0.2 to 1 percent, (5) Propylene glycol, 0.2 to 1 percent, (e) Polyvinyl alcohol, 0.1 to 4 percent and (f) Povidone, 0.1 to 2 percent.

The composition further comprises balanced salts such as ZnCl2 and MgCl2. Other inorganic materials such as HAP (tetrasodium etidronate (Monsanto)), tricalcium phosphate, dicalcium pyrophosphate, tetracalcium phosphate and octacalcium phosphate may also be included.

The composition may further comprise active agents such as any compound, composition of matter, or mixture thereof that can be delivered from the composition of the invention to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; benzodiazepine receptor agonists such as abecamil; GABA receptor modulators such as baclofen, muscimol and benzodiazepines; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrase inhibitors; diagnostic agents; antiapoptosis agents; gene therapy agents; sequestering agents; reductants such as glutathione; antipermeability agents; antisense compounds; antiproliferative agents; antibody conjugates; antidepressants; blood flow enhancers; antiasthmatic drugs; antiparasiticagents; non-steroidal anti inflammatory agents such as ibuprofen; nutrients and vitamins: enzyme inhibitors: antioxidants; anticataract drugs; aldose reductase inhibitors; cytoprotectants; cytokines, cytokine inhibitors and cytokine protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, loteprednol and triminolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

pH adjusting agents such as HCI and NaOH may also be used to adjust the pH of the final composition to between about 7.0 and about 7.4.

Tonicity agents such as those commonly used in the art may be used to bring the osmolality of the final composition to between about 240 and about 260 mOsmo/Kg.

The invention will be better understood by way of the following examples which are intended to illustrate but not limit the invention as defined by the claims contained herein. All values are weight percent unless otherwise specified.

### EXAMPLES

Of the following examples, Examples 1, 2 and 4 have been prepared. The other examples are prophetic examples.

### Example 1

| Ingredient | % W/W |
|---|---|
| Sodium Borate | 0.12 |
| Boric Acid | 0.50 |
| CMC (MV) | 0.50 |
| ZnCl₂ | 0.01 |
| MgCl₂ | 0.01 |
| Glycerin | 1.00 |
| Aloe Vera Gel | 1.00 |
| HAP (30%) | 0.10 |
| Alexidine 2HCl | 0.5 ppm-5.0 ppm |

pH = 7.0-7.4
Osmo.(mOsmo/Kg) = 240-260

### Example 2

| Ingredient | % W/W |
|---|---|
| Sodium Borate | 0.12 |
| Boric Acid | 0.50 |
| CMC (MV) | 0.10 |
| ZnCl₂ | 0.01 |
| MgCl₂ | 0.01 |
| Aloe Vera Gel | 5.00 |
| HAP (30%) | 0.10 |
| Alexidine 2HCl | 0.5 ppm-5.0 ppm |

pH = 7.0-7.4
Osmo.(mOsmo/Kg) = 240-260

### Example 3

| Ingredient | % W/W |
|---|---|
| Sodium Borate | 0.12 |
| Boric Acid | 0.50 |
| CMC (MV) | 0.10 |
| ZnCl₂ | 0.01 |
| MgCl₂ | 0.01 |
| Aloe Vera Gel | 5.00 |
| HAP (30%) | 0.10 |
| Alexidine 2HCl | 0.5 ppm-5.0 ppm |

pH = 7.0-7.4
Osmo.(mOsmo/Kg) = 220-240

### Example 4

| Ingredient | % W/W |
|---|---|
| Sodium Borate | 0.12 |
| Boric Acid | 0.50 |
| Alginate | 0.20 |
| ZnCl₂ | 0.01 |
| MgCl₂ | 0.01 |
| Aloe Vera Gel | 2.50 |
| HAP (30%) | 0.10 |
| Alexidine 2HCl | 0.5 ppm-5.0 ppm |

pH = 7.0-7.4
Osmo.(mOsmo/Kg) = 240-260

### Example 5

| Ingredient | % W/W |
|---|---|
| Sodium Borate | 0.12 |
| Boric Acid | 0.50 |
| HA | 0.20 |
| ZnCl₂ | 0.01 |
| MgCl₂ | 0.01 |
| Aloe Vera Gel | 2.00 |
| HAP (30%) | 0.10 |
| Alexidine 2HCl | 0.5 ppm-5.0 ppm |

pH = 7.0-7.4
Osmo.(mOsmo/Kg) = 240-260

### Example 6

| Ingredient | % W/W |
|---|---|
| Sodium Borate | 0.12 |
| Boric Acid | 0.50 |
| Povidone | 0.50 |
| ZnCl₂ | 0.01 |
| MgCl₂ | 0.01 |
| Aloe Vera Gel | 2.00 |
| HAP (30%) | 0.10 |
| Alexidine 2HCl | 0.5 ppm-5.0 ppm |

pH = 7.0-7.4
Osmo.(mOsmo/Kg) = 240-260

### Example 7

| Ingredient | % W/W |
|---|---|
| Sodium Borate | 0.12 |
| Boric Acid | 0.50 |
| CMC (MV) | 0.50 |
| ZnCl₂ | 0.01 |
| MgCl₂ | 0.01 |
| Glycerin | 1.00 |
| Aloe Vera Gel | 1.00 |
| HAP (30%) | 0.10 |
| Loteprednol Etabonate | 0.1-10 |
| Alexidine 2HCl | 0.5 ppm-5.0 ppm |

pH = 7.0-7.4
Osmo.(mOsmo/Kg) = 240-260

What is claimed is:

1. Compositions comprising Aloe Vera Gel preserved with a preservative selected from the group consisiting of biguanide preservative, benzalkonium chloride, sorbic acid/EDTA, H₂O₂ systems, Zinc preservative systems and stabilized hypochlorite preparations; wherein the composition is intended for ophthalmic use.

2. The composition of claim 1 wherein the biguanide preservative is Alexidine hydrochloride.

3. The composition of claim 1 further comprising a demulcent selected from the group consisting of Cellulose derivatives such as Carboxymethylcellulose sodium, Hydroxyethyl cellulose, Hypromellose, and Methylcellulose; Dextran 70; Gelatin; liquid Polyols such as Glycerin, Polyethylene glycol 300, Polyethylene glycol 400, Polysorbate 80, Propylene glycol, Polyvinyl alcohol, Povidone and mixtures thereof.

4. The composition of claim 2 further comprising a demulcent selected from the group consisting of Cellulose derivatives such as Carboxymethylcellulose sodium, Hydroxyethyl cellulose, Hypromellose, and Methylcellulose; Dextran 70; Gelatin; liquid Polyols such as Glycerin, Polyethylene glycol 300, Polyethylene glycol 400, Polysorbate 80, Propylene glycol, Polyvinyl alcohol, Povidone and mixtures thereof.

5. The composition of claim 1 further comprising an alginate.

6. The composition of claim 2 further comprising an alginate.

7. The composition of claim 3 further comprising inorganic molecules selected from the group consisting of ZnCl₂, MgCl₂, HAP, tricalcium phosphate, dicalcium pyrophosphate, tetracalcium phosphate, octacalcium phosphate and mixtures thereof.

8. The composition of claim 4 further comprising inorganic molecules selected from the group consisting of ZnCl₂, MgCl₂, HAP, tricalcium phosphate, dicalcium pyrophosphate, tetracalcium phosphate, octacalcium phosphate and mixtures thereof.

9. The composition of claim 7 further comprising an active agent.

10. The composition of claim 8 further comprising an active agent.

11. The composition of claim 9 wherein the active agent is Loteprednol etabonate.

12. The composition of claim 10 wherein the active agent is Loteprednol etabonate.
